SEQUENCE LISTING (8 sequences)

SEQ ID NO: 1 - DNA, 21 bp, Mycoplasma bovis
ccgcaagttaacttgtggtgc

SEQ ID NO: 2 - DNA, 24 bp, Mycoplasma bovis
ggccattttcttgtcagaaccacc

SEQ ID NO: 3 - DNA, 29 bp, Mycoplasma bovis
gcttttactctggtactagatggtcttgg

SEQ ID NO: 4 - DNA, 30 bp, Mycoplasma bovis
gtggcgttcttgacaatagaacaattagtg

SEQ ID NO: 5 - DNA, 26 bp, Mycoplasma bovis
gatgttcttcattgtcttttgcatcg

SEQ ID NO: 6 - DNA, 23 bp, Mycoplasma bovis
cgacgagttacaagaaagttggc

SEQ ID NO: 7 - DNA, 29 bp, Mycoplasma bovis
gaaacacctatcccagtaggtacaagatc

SEQ ID NO: 8 - DNA, 33 bp, Mycoplasma bovis
gtctacattgttcaaaatgcgacattttgtata

(19) United States
(12) Patent Application Publication
(10) Pub. No.: US 2011/0059437 A1
(43) Pub. Date: Mar. 10, 2011

(54) PCR-BASED GENOTYPING

(75) Inventor: Michael C Beck, Saint Joseph, MO (US)

(73) Assignee: BOEHRINGER INGELHEIM VETMEDICA, INC., Saint Joseph, MO (US)

(21) Appl. No.: 12/439,947

(22) PCT Filed: Sep. 7, 2007
(86) PCT No.: PCT/US07/19667

§ 371 Date: Nov. 23, 2010

Related U.S. Application Data
(60) Provisional application No. 60/824,855, filed on Sep. 7, 2006.
(60) Provisional application No. 60/867,784, filed on Nov. 29, 2006.

(51) Int. Cl. C12Q 1/68; C07H 21/04

(52) U.S. Cl. 435/6; 536/23.1

(57) ABSTRACT

A Mycoplasma bovis PCR-based genotyping method was developed that exploits the proximity of insertion sequences (IS) within the genome by using outward facing primers that selectively amplify sequences between IS elements. The method was applied to 16 field isolates of M. bovis, originating from pneumonic lung or arthritic joints, collected from the United States (Iowa or Kansas) between 2004 and 2005. The genomic fingerprints generated 14 distinct amplification profiles consisting of 4-8 fragments ranging in size from 200-3000 bp. Three isolates presented identical patterns and were isolated from two calves (one calf with pneumonic lung and the other with both pneumonic lung and arthritic joint) from a single farm during an outbreak and probably represent multiple infections with the same genotype. To demonstrate the stability of IS markers for molecular fingerprinting, 3 of the 16 field isolates were subjected to high number passage which resulted in patterns identical to the initial isolates. The results of these studies demonstrate the method can be used for simple and rapid molecular fingerprinting and differentiating M. bovis isolates with extension to epidemiology.

### RELATED APPLICATIONS

This application claims the benefit of provisional application Ser. Nos. 60/824,855, filed on Sep. 7, 2006, and 60/867,784, filed on Nov. 29, 2006, the teachings and content of which are hereby incorporated by reference herein.

### BACKGROUND OF THE INVENTION

The present application provides a novel method to determine the genetic differences between bacterial isolates that contain insertion sequence elements. Mycoplasma bovis isolates provide one example of bacterial isolates that could benefit from such a methodology. The results from such a method have many different potential applications, but essentially any application that could benefit from knowledge of genetic differences between isolates of the same bacteria would be able to take advantage of such methods. For example, the results can be used in a mixed challenge model study in order to determine which isolates are most commonly found, and which isolates appear to be the most dominant or virulent. The present application also describes a novel method for using these differences as a reference library for identifying recovered unknown isolates. As a result of answering the question of genotype, costs associated with animal testing can be reduced or eliminated by reducing study articles to those few isolates found to be the most interesting or relevant.

### DESCRIPTION OF THE PRIOR ART

There are two classes of microbial typing: phenotypic and genotypic. The criteria for the typing method is that all organisms of a species must be typeable by the method, there must be a genetic marker, high differentiation power or resolution, and it must be reproducible. The epidemiological and practical importance of molecular subtyping is for recognizing the outbreak of infection, determining the source of infection, identifying virulent strains, and monitoring vaccination programs. There are various subtyping methods, which all vary in their satisfaction of the criteria noted above. One such method is DNA sequencing, which is generally considered a relatively expensive and difficult procedure, however, it has good reproducibility. The phylogenetic resolution of DNA sequencing can differentiate family, genus, species, subspecies, and strain. This method usually takes about 2 days. 16 S rDNA sequencing can differentiate family, genus, species, and subspecies. The resolution of Amplified rDDNA Restriction Analysis, DNA-DNA reassociation, and tRNA-PCR differentiates genus, species, and subspecies. Internal Spacer Region PCR (ITS-PCR) can differentiate genus, species, and some strains. Restriction Fragment Length Polymorphism (RFLP), Low Frequencey Restriction Fragment Analysis (LFRFA), Pulsed Field Gel Electrophoresis (PFGE), Multilocus Izozyme, Whole Cell protein profiling, Amplified Fragment Length Polymorphism (AFLP), Random Amplification of Polymorphic DNA (RAPD), Arbitrary Primed PCR (APPCR), and Repetitive Extragenic Palindromic PCR (REP-PCR), all have phylogenetic resolution to differentiate species, subspecies, and strain. Of these types, PFGE has a moderate set up cost and cost per reaction, is moderately easy to use, has a normal time of 3 days, and has good reproducibility. RAPD has a moderate set up cost, a low cost per reaction, takes 3 days and is easy to use. However, this method only has moderate reproducibility. REP-PCR has a poor reproducibility, but is easy to use, takes 1 day, and has a low cost per reaction. Another method with good reproducibility is AFLP. This method has a moderate—high cost of set up, with a low—moderate cost per reaction. Additionally, it is moderately easy to use and takes 2 days.

The prior art used Retrotransposon-Microsatellite Amplified Polymorphism (REMAP), Inter-Retrotransposon Amplified Polymorphism (IRAP), RAPD, Adapter Ligated PCR, and REP-PCR to attempt to determine genetic differences. REMAP and IRAP use PCR and a single set of primers designed to microsatellite and/or retrotransposable elements in eukaryotic cells. There is no previous record of its use in bacteria, use of insertion sequences, or the use of combined primer sets. RAPD uses short random sequences of primers under low stringency PCR conditions to amplify random segments in a bacterial genome. Adapter Ligated PCR uses a restriction digestion followed by ligation of adapters to the exposed ends. A PCR reaction is set up using a specific primer with the adapter primer to achieve a pattern. REP-PCR is a direct PCR that uses primers designed to repetitive DNA elements such as Repetitive Extragenic Palindromic (REP) elements or Enterobacterial Repetitive Intergenic Consensus (ERIC) sequences.

The prior art noted above was deficient in several respects. REMAP and IRAP use a single combination of primers designed to microsat and/or retrotransposable elements from eukaryotic cells. The novel technique described herein targets insertion sequences (transposons) found in a species of bacteria. REP-PCR uses a primer(s) to PCR amplify regions between repetitive DNA elements (e.g. REP or ERIC sequences). The present technique targets the regions between insertion sequences using rationally designed out-ward facing primers. This design allows for a targeted amplification of a species. Adapter Ligated PCR has numerous procedural differences (Restriction Digestion, Ligation, Different primer combination) from the present invention. For example, this technique does not require restriction digestion or ligation.

Kalendar et al. (R. Kalendar, T. Grob, M. Regina, A. Suoniemi, A. Schulman; IRAP and REMAP: two new retrotransposon-based DNA fingerprinting techniques; TAG Theoretical and Applied Genetics, Volume 98, Issue 5, April 1999, Pages 704-711, the teachings and content of which are hereby incorporated by reference herein) describe fingerprinting from eukaryotic cells using some combination of retrotransposons and microsattalites. The present invention describes fingerprinting using insertion sequences (transposable elements) found in prokaryotes (bacteria). U.S. Pat. Nos. 5,691,136 & 5,523,217 to Lupski et al., describe fingerprinting from bacteria using repetitive DNA sequences. The present invention describes fingerprinting from bacteria using insertion sequences (transposons). Transposons are a subset of Mobile Genetic Elements (unrelated to repetitive DNA sequences). Welsh and McClelland describe arbitrary primed PCR (aka RAPD), which uses short random primers under non-stringent PCR conditions to form fingerprints (see, J Welsh and M McClelland; Fingerprinting genomes using PCR with arbitrary primers, Nucleic Acids Res. 1990 Dec. 25; 18(24): 7213-7218, the teachings and content of which are hereby incorporated by reference herein). The present invention differs as REP-PCR differs.

### SUMMARY OF THE INVENTION

The invention enables the rapid identification of bacterial strains by amplifying the DNA between insertion sequences and measuring the pattern of amplified products. Using PCR and a combination of outwardly facing primers designed against bacterial insertion sequences (transposable elements), patterns are produced that are unique to an isolate of a bacterial species. These patterns can then be compared for such things as epidemiology or phylogeny. An insertion sequence (IS) is a short DNA sequence that acts as a transposable element. IS are generally around 700 to 2500 by in length, which is relatively small compared to other types of transposable elements. They code for proteins implicated in transposition activity, wherein the proteins catalyze the enzymatic reaction allowing the IS to move. IS elements are unique to a particular species or can be shared between taxonomic groups. There are usually multiple copies of these insertion sequences, but they are located in unique locations for a specific transposable element.

One preferred method of the present invention utilizes PCR and a combination of outwardly facing primers designed against single or multiple bacterial insertion sequences. Such a method produces amplification products from adjacent IS. Once the PCR amplification is complete, the products are separated (agar gel) and banding patterns are produced, according to the molecular weight of the amplification products, that are unique to an isolate of a bacterial species. The preferred method in the present invention is to carry out multiplex PCR using outwardly facing primers. Multiplex PCR is a variant of PCR which enables simultaneous amplification of many targets of interest in one reaction by using multiple primer sets.

It is understood that the present invention lends itself to many modifications and embodiments. DNA can be extracted, obtained, or directly amplified from any whole or crudely prepared organism or microorganism. PCR reaction conditions (mix constituents, additives/enhancers, thermal conditions, etc) can be modified or optimized by those of skill in the art. Generally PCR reactions will be carried out using a master mix kit (e.g. from Qiagen or Biorad); 50-500 nM, more preferably 100-450 nM, still more preferably 150-400 nM, even more preferably 200-350 nM, still more preferably 250-325 nM, and most preferably about 300 nM of each primer; and 0.5-10 ng, more preferably 0.6-8 ng, still more preferably 0.7-6 ng, even more preferably 0.8-4 ng, still more preferably, 0.85-2.5 ng, even more preferably, 0.9-1.75 ng, still more preferably 0.95-1.25 ng, and most preferably about 1 ng of template DNA. Intial thermal cycling conditions during the wake-up step were 90-100° C., more preferably 91-99° C., even more preferably 92-98° C., still more preferably 93-97° C., even more preferably 94-96° C., and most preferably about 95° C. The wake-up step can last from 5-25 minutes, more preferably from 10-20 minutes, even more preferably from 12-18 minutes, still more preferably from 14-16 minutes, and most preferably for about 15 minutes. Next, 25-50, more preferably 30-45, still more preferably, 32-40, even more preferably 34-36, and most preferably about 35 cycles, each comprising from 4, and more preferably 5, cycle steps, are run. The first cycle step temperature is generally between 89-99° C., more preferably 90-98° C., even more preferably 91-97° C., still more preferably 92-96° C., even more preferably 93-95° C., and most preferably about 94° C. The first cycle step time period is generally from 15 seconds to 1 minute, more preferably 20 seconds to 50 seconds, even more preferably 25 seconds to 40 seconds, and most preferably about 30 seconds. The second cycle step temperature is generally between 51-61° C., more preferably 52-60° C., even more preferably 53-59° C., still more preferably 54-58° C., even more preferably 55-57° C., and most preferably about 56° C. The second cycle step time period is generally from 15 seconds to 2 minutes, more preferably 30 seconds to 110 seconds, even more preferably 45 seconds to 100 seconds, still more preferably from 60 to 95 seconds, even more preferably from 75-93 seconds and most preferably about 90 seconds. The third cycle step temperature is generally between 67-77° C., more preferably 68-76° C., even more preferably 69-75° C., still more preferably 70-74° C., even more preferably 71-73° C., and most preferably about 72° C. The third cycle step time period is from 30 seconds to about 7 minutes, more preferably 35 seconds to about 6 minutes, even more preferably 40 seconds to about 5 minutes, still more preferably between about 45 seconds and 4 minutes, even more preferably between about 50 seconds to about 3 minutes, still more preferably between about 55 seconds to about 150 seconds, even more preferably between about 1 minute and about 130 seconds, and most preferably about 2 minutes. The fourth cycle step (for final extension) temperature is between 67-77° C., more preferably 68-76° C., even more preferably 69-75° C., still more preferably 70-74° C., even more preferably 71-73° C., and most preferably about 72° C. The fourth cycle step time period is from about 1 minute to about 10 minutes, more preferably 2 minutes to about 9 minutes, even more preferably from about 3 minutes to 8 minutes, still more preferably from about 3.25 minutes to about 7 minutes, even more preferably from about 3.5 minutes to about 6 minutes, still more preferably from about 3.75 minutes and about 5 minutes, and most preferably about 4 minutes. Optionally, the PCR reaction can have a hold time at a reduced temperature between about 2 and 10° C., more preferably between about 2.5 and 9° C., even more preferably between about 3 and 8° C., still more preferably between about 3.25 and 7° C., even more preferably between about 3.5 and 6° C., still more preferably between about 3.75 and 5° C., and most preferably about 4° C. Amplified products are then detected using any conventional process, such as agarose gel with ethidium bromide. When agarose gel is used, the agarose can be between 2-6%, more preferably 3-5%, and most preferably about 4%. Temperature conditions during the detecting step when using agarose gel are generally around room temperature. Additionally, when using agarose gel, imaging is done under ultraviolet light and the gels can generally run between 25 and 75 minutes, more preferably between 35 and 65 minutes, still more preferably between 45 and 55 minutes, and most preferably for about 50 minutes. Invitrogen E-gel is one example of a suitable agarose gel.

Any combination of outwardly facing IS primers can be used for purposes of the present invention. Exmples of primers include those identified as SEQ ID Nos. 1-8, although it is understood that these are representative in nature and other primers can be designed by those of skill in the art. Moreover, it is understood that these primers can be further modified to include additional or fewer nucleotides upstream and/or downstream of the specifically defined sequences herein. Within the defined sequences herein, it is further understood that those of skill in the art can modify these sequences such that they are not 100% identical with those specified herein, yet still operate in a similar manner. Examples of some such modifications include mutations changes in the nucleotide sequence that have no effect on the amino acid encoded by the nucleotide triplet and mutations or alterations that have no effect on the function of the PCR product produced. Primers can also be designed for other insertion sequences found in the same or other bacteria (i.e., in addition to the Mycoplasma bovis IS used herein for proof of concept). Primers designed to conserved IS elements found in more than one species (or higher taxanomic layer) or outwardly-facing IS specific primers with gene specific primers can be used. Primers can further be modified with flourophores (fluorescent dyes) or any other tagging method to aid in identification. Digestion (restriction digestion, chemical cleavage, etc) of the PCR product with follow-on detection and analysis can be used. The detection/characterization of amplicons can be accomplished by alternate methods (intercalating dyes, melt curve analysis, capillary gel electropheresis, microfluidic chips, alternate gel methods [PAGE/agarose/DGGE/TGGE,etc], Gel/CE sequencing, etc). Techniques to increase sample throughput or analysis (ex. Robotic automation for liquid transfer) of any step or steps in the invented process can be used. Virtual patterns specific to the invention can be generated using computer software. The amplicon can be compared to a library of known fingerprints to determine the identity of bacteria. The present invention can be used as a tool for diagnosing a suspected bacterial disease, recognizing outbreak of infection, determining the source of infection, and to monitor vaccination programs. Computer software can be used for databasing or comparing gel patterns derived from the invention.

In yet another embodiment of the present invention, a kit is provided. At a minimum, such a kit would comprise at least one set or pair of primiers designed similar to those disclosed herein, namely to IS sequences and a set of instructions or protocol to follow. Of course, use of such a kit would require the user to have access to the equipment necessary to run the PCR protocol and detect the amplification products. The specific primers used in the present application are particularly applicable to the fingerprinting or identification of bacterial isolates of Mycoplasma, and more specifically, to M. bovis. Preferably, more than one set of primers will be included such that a multiplex PCR protocol can be employed. The user would provide a thermocycler and equipment to detect the amplification products, preferably a gel and apparatus for running the PCR product onto a gel. Such a kit would be to help determine the particular strains of a bacterial isolate present in a mixed infection. As shown herein, M. bovis is well suited for such methodology and such a kit. The protocol or instructions included with such a kit would instruct the user to obtain a sample from a sick animal, isolate the bacteria, grow colonies on plates which agar that suppress the growth of microorganisms except the bacteria of interest, (as shown herein by the use of Mycoplasma. A colony would then be selected and that sample used for the PCR multiplex reaction. The kit could be offered with any quantity of primer pairs, however, a 3 plex or 4 plex system is preferred. Use of such a kit could also determine the source of an outbreak as well as whether the source was from a vaccine or if the strain was wild type. In a preferred embodiment, the kit could be specific to M. bovis. A more complex or complete kit would include all of the components of the basic kit described above, and any combination of specialized software or apparatus which could more accurately carry out the PCR reaction. The protocol included in such a kit would include DNA extraction, PCR, detection, and read out. Potentially, in an all-inclusive kit, specialized methods, software, or apparatus could be provided for each of the steps. In another embodiment the kit would be accompanied by a master mix particularly designed for the PCR reaction.

### DESCRIPTION OF THE DRAWING FIGURES

FIG. 1 is photograph of a gel showing the unique banding patterns of five different M. bovis field isolates;

FIG. 2 is a photograph of a gel showing how isolates of unknown identity can be compared to isolates of known identity based on the banding patterns created using the IS-PCR methods of the present invention;

FIG. 3 is schematic drawing illustrating the differences between single IS PCR and multiplex IS PCR;

### DETAILED DESCRIPTION OF THE PREFERRED EMBODIMENTS

The following examples set forth preferred embodiments of the present invention. These examples are for illustrative purposes only and the disclosure herein should not be construed as a limitation upon the scope of the present invention.

### Example 1

This example isolates, amplifies and detects the DNA for use with methods of the present invention. Mycoplasma sp. isolates were used in the studies. Isolates were obtained from in-house sources or field isolates obtained from infected animals. Isolates were grown using a combination of mycoplasma selective agar and broth for 1-7 days. To isolate DNA, broth cultures were spun and pelleted. DNA from the pellet was then extracted (using the Qiagen DNeasy Tissue Kit and resuspended in molecular grade water). Genomic DNA was quantitated using Picogreen (Invitrogen). Primers were designed based on the known insertion sequences (transposable elements) present in the bacterial genome (Mycoplasma bovis). Outwardly facing primers were manually selected from the element ends (excluding the terminal repeat regions) at a Tm of 55-58C. PCR reactions were then carried out using a multiplex PCR master mix (Qiagen Multiplex PCR Kit). The reactions contained 1× Master mix, 300 nM of each primer and ing of template DNA. Thermal cycling conditions were 95 C for 15 minutes, 35 cycles of 94 C for 30 seconds, 56.1 C for 90 seconds, 72 C for 2 minutes, with a final extension of 72 C for 4 minutes and a 4 C hold. The amplified products were separated on a 4% agarose gel with ethidium bromide (Invitrogen E-gel), run for 50 minutes at room temperature and imaged under UV light.

### Example 2

This Example demonstrates the existence of unique genetic fingerprints between strains of M. bovis. Five field isolates were grown and DNA isolated according to the above protocol. 2-5 ng of DNA from each isolate was amplified according to the above protocol using a multiplex of 4 sets of IS primers identified as SEQ ID Nos 1-8. The amplified products were separated on a Invitrogen E-gel 4% agarose gel containing ethidium bromide (according to manufacturer) for 50 minutes and visualized under UV light. All isolates produced unique patterns. The patterns were reproducible using independent aliquots under the sample PCR reaction conditions. Results are shown below in FIG. 1.

### Example 3

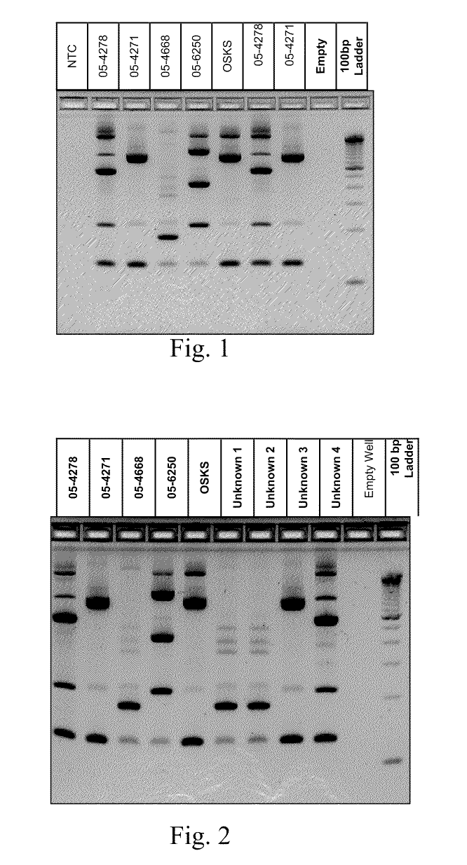

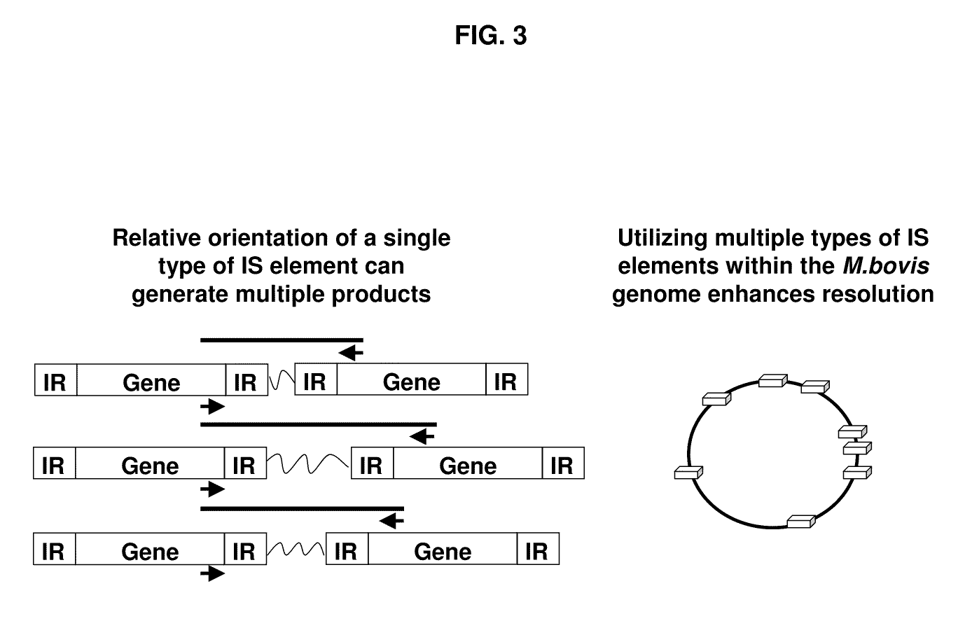

This Example identifies unknown strains of M. bovis against a library of known M. bovis strains. M. bovis from infected animals were isolated, DNA extracted and amplified (ing) according to the above protocol using a multiplex of 4 sets of IS primers identified as SEQ ID Nos. 1-8. The amplified products were separated on a Invitrogen E-gel 4% agarose gel containing ethidium bromide (according to manufacturer) for 50 minutes and visualized under UV light. As shown below in FIG. 2, all isolates recovered from infected animals produced an amplification pattern that matched one of the patterns found in a library of suspected strains. Unknown strains 1 and 2 matches 05-4668, unknown strain 3 matches 05-2471 and unknown strain 4 matches 05-4278.

### Example 4

This example determines which isolate(s) are recovered with the highest frequency from a challenge mix of “best” M. bovis isolate candidates. Information from this experiment can be used to develop a good challenge model.

### Materials and Methods

Five clostrum-deprived beef calves were purchased from Midwest Veterinary Services Inc., Oakland, NE. All calves were screened for previous M. bovis exposure by serology and nasal swabs. All calves tested negative for M. bovis and met the protocol inclusion criteria which included an age range of 10+/−2 weeks of age at the initiation of the study, age-appropriate weight, healthy, and no history of receiving a commercial or autogenous M. bovis vaccine.

A mixture of five isolates of M. bovis were tested in this study. Isolates used for challenge were obtained from naturally occurring disease outbreaks and demonstrated the highest recovery from a previous mixed challenge study. The isolates were identified and designated as 05-249, 05-4278, 05-4271, OSKS and 24466-192. Verification of identity was made by growth on Mycoplasma selective medium, real-time PCR and IS-multiplex PCR (as described herein).

Each challenge isolate was separately grown in 100 ml of Friis media supplemented with 10% yeast extract and 20% horse serum. The cultures were grown 20±2 hours at 37° C. after inoculation with 3e5 CFU seed culture. Equivalent amounts of each M. bovis isolate were pooled into a group. The pooled challenge material was then aliquoted and held on ice until challenge.

Aliquots of pooled group material were analyzed for M bovis by serial dilution on PPLO agar plates and grown for 5 days at 37° C./5% CO2. In addition, blood agar quality control plates were incubated for 10 days at 37° C./5% CO2 and showed no signs of contaminating growth.

Calves were randomly assigned to one of two pens. On the day of challenge, each animal was anesthetized and an endoscope was placed in the trachea and guided to the right bronchial tree into the bifurcaiton of the medial lobe of the lung. A 25 ml dose of test/control article (3.8E+09 total CFU) was administered followed by a 25 ml sterile PBS wash.

Nasal swab and blood samples were collected at 5 days pre-challenge, on the day of challenge, and at 7 and 14 days post challenge. On day 14 post challenge, the animals were necropsied for gross pathology and antemortem samples were collected from tonsils, joints, and lung tissue. Blood was also collected aseptically from a jugular vein from each calf into a 12.5 mL Serum Separator Tube (SST).

DNA was extracted from swabs from each calf and was tested using real-time PCR using primers and probes specific for the uvrC gene of M. bovis. Results of real-time PCR were expressed as either positive or negative for M. bovis detection. Additionally, DNA was extracted from isolates recovered from lung tissue then tested by IS-multiplexPCR using primers specific for M. bovis insertion sequences developed at Boehringer Ingelheim Vetmedica, Inc. and listed herein as SEQ ID Nos. 1-8.

### Results and Conclusion

### Clinical Observations

All calves were generally observed daily from day of arrival to DPC (days post challenge)-1 and clinical observations were made daily from DPC-1 to euthanasia and necropsy. Calf 5459 did show some swelling in the left hock and right carpal joints during this phase of the trial. It should be noted that this calf exhibited these symptoms upon arrival to study site. The calves all remained normal through DPC-6, and then clinical signs were observed from DPC7 remained through the conclusion of the trial. Coughing, nasal discharge, depression, and diarrhea were clinical observations noted during this phase. Lung severity was most notable in ID 5460, being 24%, while ID 5458 observed no lung lesions. All calves but ID 5457 showed growth of M. bovis by DPC 14. A table of individual results is below:

TABLE 1

| | DPC | | | | | | | | | | |
|---|---|---|---|---|---|---|---|---|---|---|---|
| | −5 | | | 0 | | | 7 | | | 14 | |
| | Nasal | | Serum | Nasal | | Serum | Nasal | | Serum | Nasal | |
| ID | Micro | PCR | ELISA | Micro | PCR | ELISA | Micro | PCR | ELISA | Micro | PCR |
| 5457 | 0 | 0 | 1 | 0* | 0 | 1 | 0 | 1 | 3 | 0 | 1 |
| 5458 | 0 | 0 | 0 | 0 | 0 | 0 | 1 | 1 | 1 | 1 | 1 |
| 5459 | 0 | 0 | 1 | 0 | 0 | 1 | 0* | 1 | 3 | 1 | 1 |
| 5460 | 0 | 0 | 0 | 0 | 0 | 0 | 1 | 1 | 1 | 1 | 1 |
| 5461 | 0 | 0 | 0 | 0 | 0 | 0 | 1 | 1 | 2 | 1 | 1 |

| | DPC | | | | | | | | | | |
|---|---|---|---|---|---|---|---|---|---|---|---|
| | 14 | | | | | | | | | | |
| | | | Lung | | | | | | | | |
| | Tonsil | | | | % | Lesion | | | Joint | | Serum |
| ID | Micro | PCR | Micro | PCR | Lung | Severity | IHC | Comment | Micro | PCR | ELISA |
| 5457 | 1 | 1 | 1 | 1 | 6 | 0 | 0 | 0 | 0 | 1 | 4 |
| 5458 | 1 | 1 | 1 | 1 | 0 | 0 | 0 | 0 | 1 | 1 | 2 |
| 5459 | 1 | 1 | 1 | 1 | 18 | 1 | 1 | 1 | 0 | 0 | 3 |
| 5460 | 1 | 1 | 1 | 1 | 24 | 2 | 2 | 1 | 0 | 0 | 3 |
| 5461 | 1 | 1 | 1 | 1 | 14 | 2 | 2 | 1 | 0 | 1 | 3 |

KEY:
Micro
0 = No M. bovis growth
0* = growth of other bacteria
1 = Growth of M. bovis
Lesion Score
0 = None
1 = Mild
2 = Moderate
3 = Severe
ELISA
0 = Negative
1 = Positivity to 1.75x
2 = 1.75x to 2.3x
3 = 2.3x to 3.0x
4 = >3.0x
PCR
0 = Negative
1 = Positive
0 = Single
Lung Comment
0 = None
1 = Bacterial pneumonia
IHC
0 = None
1 = Scattered macrophages positive
2 = Clusters of macrophages positive, multiple sites, scattered airways
3 = Abundant positive staining of Macrophages and airway debris

### Microbiology

M. bovis was recovered from all tonsil swabs and lung tissue sampled on DPC-14. In addition, M. bovis was recovered from joint swabs of calf ID 5458. No clinical signs were present until DPC 7. Calves with IDs 5457, 5458, and 5461 only showed the clinical sign of coughing, while calf ID 5460 showed the widest range of clinical symptoms including coughing, nasal discharge, depression, and gauntness. The only calf to exhibit diarrhea was ID 5459. The following table provides a summary of culture, PCR, Serology, and Lung Pathology:

TABLE 2

| | DPC | | | | | | | | | | | | | | | |
|---|---|---|---|---|---|---|---|---|---|---|---|---|---|---|---|---|
| | −1 | 0 | 1 | 2 | 3 | 4 | 5 | 6 | 7 | 8 | 9 | 10 | 11 | 12 | 13 | 14 |
| | ID | | | | | | | | | | | | | | | |
| | 8/1 | 8/2 | 8/3 | 8/4 | 8/5 | 8/6 | 8/7 | 8/8 | 8/9 | 8/10 | 8/11 | 8/12 | 8/13 | 8/14 | 8/15 | 8/16 |
| 5457 | 0 | 0 | 0 | 0 | 0 | 0 | 0 | 0 | 0 | 0 | 1 | 1 | 0 | 0 | 1 | 1 |
| 5458 | 0 | 0 | 0 | 0 | 0 | 0 | 0 | 0 | 0 | 0 | 0 | 0 | 0 | 1 | 0 | 0 |
| 5459 | 0 | 0 | 0 | 0 | 0 | 0 | 0 | 0 | 7 | 7 | 1 | 1 | 1 | 1, 7 | 1, 3, 8 | 1, 3 |
| 5460 | 0 | 0 | 0 | 0 | 0 | 0 | 0 | 0 | 2 | 2 | 1, 3 | 3 | 0 | 1, 2, | 1, 2, 3, | 1, 2, |
| | | | | | | | | | | | | | | 3 | 8 | 3 |
| 5461 | 0 | 0 | 0 | 0 | 0 | 0 | 0 | 0 | 0 | 0 | 1 | 1 | 0 | 1 | 0 | 0 |

Key Clinical Signs:
1 = Coughing
2 = Nasal Discharge
3 = Depression
4 = Anorexia
0 = none observed
5 = Rapid respiration
6 = Swollen joint
7 = Diarrhea
8 = Other (gaunt)

Nasal swabs were collected from all calves on DPC 0, 7, and 14. All samples were evaluated by real-time PCR. All animals were negative from DPC 0 nasal swabs. All animals were positive from DPC7 nasal swabs and all DPC 14 samples (nasal, tonsil, lung). In addition, joint swabs were collected from the left hock of all animals with 5457, 5458, and 5461 were positive for M. bovis. Results from individual animals can be found in Table 2 above.

Genotype banding patterns from each lung isolate matched one of the original challenge isolates. Table 3 is a summary showing the percentage of genotypes recovered from lung samples matching the original challenge isolates. Every calf exhibited at least two different isolates, with Calf ID 5461 exhibiting 4 different isolates. None of the calves exhibited isolate 05-4278 and only ID 5461 exhibited isolate 05-4271. All calves exhibited isolates 05-249 and 2466-192. Genotypes differing from the original challenge isolates were not recovered:

TABLE 3

| | |
|---|---|
| Calf | Genotype - % |

| ID | 05-249 | 05-4278 | 05-4271 | OSKS | 24466-192 |
|---|---|---|---|---|---|
| 5457 | 25 | 0 | 0 | 0 | 75 |
| 5458 | 44 | 0 | 0 | 12 | 44 |
| 5459 | 0 | 0 | 0 | 19 | 81 |
| 5460 | 50 | 0 | 0 | 43 | 7 |
| 5461 | 31 | 0 | 6 | 25 | 38 |

### Serology

Serum was collected on DPC 0, 7, 14, and then tested in the Biovet M. bovis ELISA to monitor the serological response to M bovis. Seroconversion was scored according to grouped multipliers of positivity ODs. The results are displayed in Table 2 above.

### Lung Pathology

At necropsy (DPC-14), lungs were collected and observed for lesions associated with M. bovis. Animals challenged with M. bovis exhibited a common pathological feature of blue discoloration and consolidation. The area where the calves received the inoculums (the right bronchial tree) showed the highest incidence of gross pathology. The results show a percentage using a scoring system reflecting the percentage of the lotal lung with gross pathology associated with M. bovis. ID 5460 showed the greatest percentage lung involvement (24%) while ID 5458 showed the least (0%). These results are shown in Table 4 below:

In conclusion, three isolates were frequently recovered from a “best” mix challenge of M. bovis in CD calves. Direct lung challenge of a mix of M. bovis isolates resulted in clinical, lung pathology and pathogen recovery consistent with the disease.

What is claimed is:

1. A method of genotyping bacterial isolates from a sample comprising the steps of:
obtaining bacterial DNA from said sample;
providing at least one pair of outwardly facing primers, each of said primers being located near the terminal end of an insertion sequence element;
amplifying said primers using PCR to produce amplification products; and
detecting the banding patterns of said amplification products.

2. The method of claim 1, said sample being obtained from an animal.

3. The method of claim 2, said animal being suspected of having a bacterial infection.

4. The method of claim 1, said detecting step comprising the step of using an agarose gel.

5. The method of amplifying step comprising 25-50 PCR cycles.

6. The method of claim 5, each of said PCR cycles having a plurality of substeps.

7. The method of claim 7, said amplification step using a master mix kit.

8. The method of claim 5, further comprising a wake-up step at a temperature of about 90-100° C. and lasting from about 5 to 25 minutes.

9. The method of claim 6, said plurality of substeps including at least 4 substeps.

10. The method of claim 9, the first substep having a temperature between 89 to 99° C. and lasting for 15 seconds to 1 minute.

11. The method of claim 9, said second substep having a temperature between 51 to 61° C. and lasting for 15 seconds to 2 minutes.

12. The method of claim 9, said third substep having a temperature between 67 to 77° C. and lasting for 30 seconds to about 7 minutes.

13. The method of claim 9, said fourth substep having a temperature between 67 to 77° C. and lasting for 1 to 10 minutes.

14. The method of claim 9, further comprising a hold step after the fourth substep, said hold step having a temperature between 2 and 10° C.

15. The method of claim 1, said primers having at least 80% sequence homology with or encoding the same amino acid as a sequence selected from the group consisting of SEQ ID Nos. 1-8.

16. A DNA sequence having at least 80% sequence homology with or encoding the same amino acid aas a sequence selected from the group consisting of SEQ ID Nos. 1-8.

17. A method of identifying an unknown bacterial strain comprising the steps of:
obtaining bacterial DNA from said strain;
providing at least one pair of outwardly facing primers, each of said primers being located near the terminal end of an insertion sequence element;
amplifying said primers using PCR to produce amplification products;
detecting the banding patterns of said amplification products; and
comparing said banding patterns with a library of banding patterns from known bacterial strains.

18. The method of claim 17, said bacterial strain being M. bovis.

19. The method of claim 17, said detecting step using agarose gel.

20. The method of claim 17, said primers having at least 80% sequence homology with or encoding the same amino acid as a sequence selected from the group consisting of SEQ ID Nos. 1-8.

21. A kit for identifying bacterial strains comprising:
at least one pair of outwardly facing primers, each of said primers being located near the terminal end of an insertion sequence element; and
protocol for amplifying said primers using PCR to produce amplification products that can be detected and used to identify the bacterial strain present.

22. The kit of claim 21, said primers having at least 80% sequence homology with or encoding the same amino acid as a sequence selected from the group consisting of SEQ ID Nos. 1-8.

23. The kit of claim 21, further comprising a component selected from the group consisting of: a PCR master mix, a thermocycler, a detection system, specialized software for running the PCR reaction or detection system, and combinations thereof.
